(19) United States
(12) Patent Application Publication
(10) Pub. No.: US 2026/0041665 A1
(43) Pub. Date: Feb. 12, 2026

(54) J-Tp Protection

(71) Applicant: HUYABIO International, LLC, San Diego, CA (US)

(72) Inventors: Gary T. Elliott, Windsor, CA (US); Suzanne J. Romano, Carlsbad, CA (US); Mireille G. Gillings, San Diego, CA (US); Jerome B. Riebman, San Diego, CA (US)

(21) Appl. No.: 19/295,253

(22) Filed: Aug. 8, 2025

Related U.S. Application Data
(60) Provisional application No. 63/680,679, filed on Aug. 8, 2024.

(51) Int. Cl. A61K 31/4025; A61P 9/06

(52) U.S. Cl. CPC A61K 31/4025; A61P 9/06

(57) ABSTRACT

Provided herein are compositions and methods protection against QTc prolongation. In some embodiments, the compositions and methods comprise administering a compound having one or more of early sodium ion channel blocking, lates sodium ion channel blocking, and L-type calcium channel blocking activity. In some embodiments, the compound is sulcardine or a pharmaceutically acceptable salt thereof, such as sulcardine sulfate or sulcardine mono-edisylate.

### CROSS-REFERENCE

This application claims the benefit of priority of U.S. Provisional Application No. 63/680,679 filed Aug. 8, 2024, the contents of which are incorporated herein by reference in their entirety.

### FIELD

The present teachings relate to compositions and methods for protecting against pathological prolongation of the QT interval.

### BACKGROUND

Iatrogenic prolongation of the QT interval can lead to heart arrythmias, including ventricular tachycardia and Torsades de Pointes (TdP). Pathological QT prolongation has led to withdrawal of drugs from the market (e.g., terfenadine and prenylamine). QT prolongation often arises due to delayed repolarization of ventricular cardiomyocytes. Special attention is directed to cardiac antiarrhythmic drugs, such as procainamide, quinidine, and disopyramide, due to their known and suspected effects on cardiac ion channels. As a result of the experience with terfenadine and prenylamine, pharmaceutical regulators in various countries now routinely require new drug applications to include QT prolongation studies in all phases of drug development.

U.S. Pat. Nos. 8,541,464 and 8,637,566 (each of which is incorporated herein by reference in its entirety) describe the activity of N-[4-hydroxy-3,5-bis(1-pyrrolidinylmethyl)benzyl]-4-methoxybenzenesulfonamide (hereinafter “sulcardine”) and its pharmaceutically acceptable salts, in addition to various uses and methods of administering sulcardine in therapeutically effective amounts to subjects in need thereof. U.S. Pat. Nos. 11,364,223 B2, 11,883,380 B2, and 11,813,245 B2 teach methods and compositions comprising sulcardine sulfate for the treatment of atrial fibrillation. U.S. Pat. Nos. 11,426,382 B2 and 11,020,374 teach, e.g., edisylate salts of sulcardine (i.e., 1,2-ethanedisulfonate salts of sulcardine). Each of these patents is incorporated herein by reference in its entirety.

Chen et al. reports the pharmacokinetics profiles of sulcardine in humans when administered orally. See Chen et al., “Pharmacokinetics, safety, and tolerability of sulcardine sulfate: an open-label, single-dose, randomized study in healthy Chinese subjects,” Fundamental & Clinical Pharmacology. 31 (2017) 120-125.

There remains a need for developing formulations and methodology for alternative administration of sulcardine in humans to achieve different but desirable pharmacokinetic and efficacy profiles.

### SUMMARY

Some embodiments presented herein provide a method of protecting against QTc prolongation in a human subject, comprising administering to the human subject an effective amount of a compound, wherein the compound has a JTp interval shortening activity and the human subject has an electrocardiogram (ECG) having a QTc. The reduction in JTp interval arises from a decrease in duration of the early repolarization period of cardiomyocytes in the treated subject.

Some embodiments presented herein provide a method of protecting against QTc prolongation in a human subject, comprising administering to the human subject an effective amount of a compound, wherein the compound inhibits one or both of a late sodium inward ion current and an L-type calcium current in cardiomyocytes, thereby shortening a JTp interval.

Thus, some embodiments presented herein provide a method of treating arrhythmia in a human subject, wherein the method ensures that the human subject does not experience a pathological lengthening of the QT interval. This is achieved by administering to the subject a compound that shortens the JTp interval to compensate for the tendency to lengthen the QT interval due to inhibition of repolarizing cation currents by the same or another drug.

These and other features, aspects and advantages of the present teachings will become better understood with reference to the following description, examples and appended claims.

### BRIEF DESCRIPTION OF THE FIGURES

FIG. 1 shows the QTc duration effects after a 30-minute IV infusion of 200 mg, 300 mg, and 500 mg of sulcardine (as sulcardine sulfate) at 0 minutes, 30 minutes, 1.5 hours and 2.0 hours after start of administration in patients in acute atrial fibrillation (AF). The change in QTcF seen with sulcardine after a 30-minute IV infusion is dose-dependent and modest. QTcF recovers quickly after completion of infusion. The depicted thresholds of concern for pro-arrhythmic risk due to QT prolongation are QTc prolongation of greater than 60 ms and absolute QTc (or simply “QTc”) of greater than 500 ms. All sulcardine doses are expressed in milligrams (mg) of the sulfate salt of sulcardine; however, free base form of sulcardine is responsible for the drug's pharmacological activity (ion channel blockade) and is the active form when dosed clinically.

FIG. 2 compares the change in QTcF after IV infusion of sulcardine (as sulcardine sulfate) and Acesion Pharma's AP30663. The change in QTcF observed with sulcardine after a 30-minute IV infusion compares favorably to that seen with AP30663 30-minute IV infusion. The two lower doses of sulcardine, which are effective for treating acute AF, had minimal QTc changes, which were below the lowest dose of AP30663. The higher dose of AP30663 was more effective than the lower dose but had greater effects on QTc.

FIG. 3 shows sulcardine levels in individual acute AF patients after 30-minute IV infusion of 500 mg of sulcardine (as sulcardine sulfate). Sulcardine levels vary widely in AF patients, even within single dose levels. Conversion from AF to sinus rhythm occurred at all dose levels tested and, within each dose cohort, at qualitatively low, medium and high plasma concentrations.

FIG. 4 shows the electrocardiogram (ECG) intervals (y-axis, dECG, 90% confidence interval, milliseconds) versus sulcardine blood plasma concentration (nanograms of sulcardine free base per milliliter of blood plasma) results from a Phase 1 study in healthy volunteers who were in sinus rhythm (SR). Means are shown in dark lines with error bars shown in shaded areas around the mean lines. Electrocardiogram parameter changes proportional to sulcardine plasma concentrations were observed, including a modest QTcF prolongation. “Observed dQTcF”, middle slope. Sulcardine strongly reduced JTpc (“Observed dJTpc,” bottom slope.) The decrease in QTc due to the JTpc reduction was as much as 21 msec at the highest concentrations (in other words, the QTc increase would have been 21 msec higher if not for the contribution of the reduced JTpc).

FIG. 5 shows a similar analysis for patients what in acute AF, and includes the effect of prolongation of the T peak to T end (TpTe). In FIG. 6, sulcardine plasma concentration is shown on the X-axis (ng/mL) and change in ECG features relative to placebo (dECG) with their 90% confidence intervals (90% CI) in milliseconds (ms). As can be seen, the dTpTe trends slightly upward with increased plasma concentration of sulcardine. As observed in the healthy volunteers in SR, the increased QTc is strongly counteracted by the steep decrease in dJTpc.

FIG. 6 shows an idealized ECG trace with labels for the QRS complex, the J point, the T wave and its peak (Tpeak), the end of the T peak (Tend), and how they map to the various phases of the action potential for a typical cardiomyocyte. Figure adapted from Lester et al., Int. J. Mol. Sci. 20;1324 (2019).

FIG. 7 depicts the QT interval of an idealized electrocardiogram (ECG) (P and U waves not shown). Also illustrated are the JTp and TpTe intervals.

FIG. 8 depicts an idealized ventricular myocyte action potential. DETAILED

### DESCRIPTION

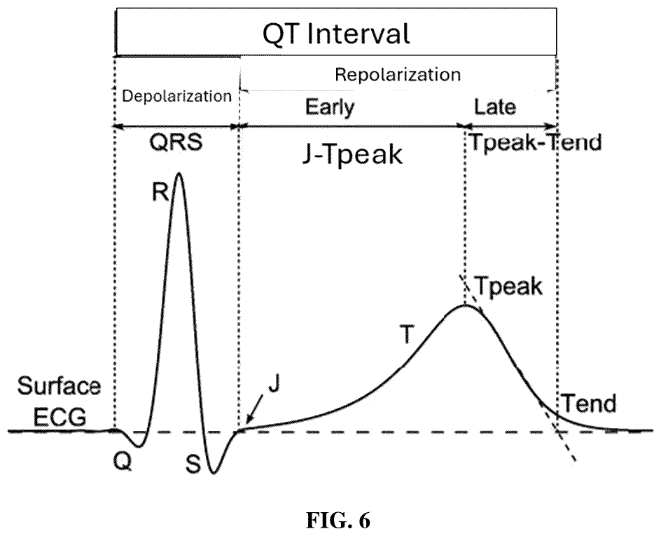

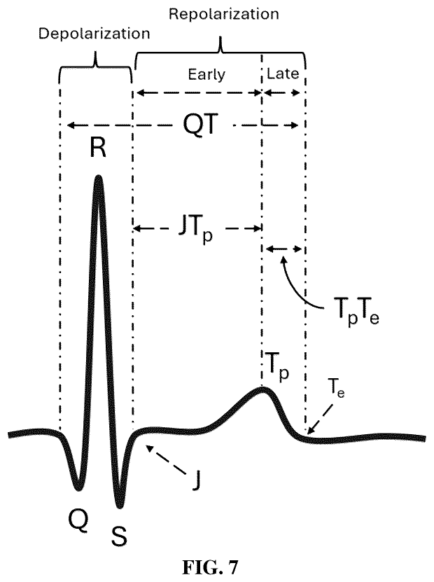

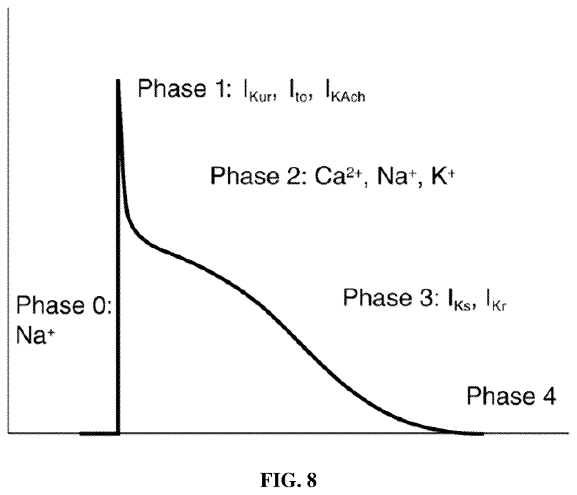

Drugs for treating arrhythmia in humans have been limited by their propensity to cause QT prolongation. The problem can be visualized with reference to FIGS. 6-8. FIG. 6 shows a portion of an idealized electrocardiogram (ECG) trace with labels for the QRS complex, the J point, the T wave and its peak (Tpeak), the end of the T peak (Tend), and how they map to the various phases of the action potential for a typical cardiomyocyte. FIG. 7 depicts the QT interval of an idealized ECG (P and U waves not shown). Also illustrated are the JTp and TpTe intervals. FIG. 8 depicts an idealized ventricular myocyte action potential. Phase 0 begins with rapid, inward Na⁺ current, followed by slower Na⁺ inward currents. Phase I begins with closing of the inward Na⁺ channels and opening of transient outward (to) K⁺ ion channels (Ito) when the membrane potential reaches its maximum positive voltage. This transient outward K⁺ current, Ito, reduces the membrane potential. As the membrane potential starts to approach 0 mV, long opening Ca²⁺ (I_{Ca.L}) and late Na⁺ (I_{Na. late}) channels open, permitting influx of Ca²⁺ and Na⁺ ions, respectively. These currents balance the efflux of repolarizing K⁺ ions (I_{Kr}), leading to a membrane potential plateau throughout Phase 2. Phase 3 begins as the outward K⁺ currents (rapidly delayed rectifier, I_{Kr}, and slowly activating delayed rectifier current, I_{Ks}) through rapid and slow delayed rectifier K⁺ channels (Kr and Ks, respectively) exceed the inward Ca²⁺ and Na⁺ currents. Eventually the membrane potential returns to its minimal, negative, potential, eventually entering Phase 4, which is characterized by negative membrane potential due to K⁺ and Na⁺ being held in balance by the Na⁺/K⁺ ATPase pump and K⁺ rectifier current (I_{KI}) and calcium ions are pumped out of the cell by the Ca²⁺ ATPase pump and the sodium-calcium exchanger. QT interval prolongation arises from inhibition of repolarizing cation currents, e.g., I_{Kr} and I_{Ks}, which delays repolarization.

### Definitions

Sulcardine sulfate (HBI-3000) is a multi-ion channel blocker in development for treating atrial fibrillation (AF). The free base of sulcardine has a chemical name of N-[4-hydroxy-3,5-bis(1-pyrrolidinylmethyl)benzyl]-4-methoxybenzenesulfonamide, and has the following structure:

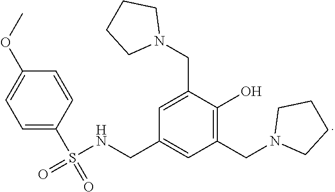 [SMILES: COc1ccc(S(=O)(=O)NCc2cc(CN3CCCC3)c(O)c(CN3CCCC3)c2)cc1]

Although the form of sulcardine used in the studies described herein, HBI-3000, is sulcardine sulfate, in some embodiments, a different pharmaceutically acceptable salt of sulcardine may be used. Such pharmaceutically acceptable salts include ethane-1,2-disulfonic acid (edisylate), naphthalene-1,5-disulfonic acid, 1-hydroxy-2-naphthoic acid, naphthalene-2-sulfonic acid, dihydro sulfonic acid, hydrochloric acid, or hydrobromic acid. In some embodiments, the pharmaceutically acceptable salt is ethane-1,2-disulfonic acid. In some embodiments, the pharmaceutically acceptable salt is mono-ethane-1,2-disulfonic acid (mono-edisylate).

In some embodiments, the sulcardine sulfate is sulcardine sulfate trihydrate.

As used herein and unless otherwise specified, “Cmax” refers to maximum plasma concentration, generally expressed in nanograms per milliliter (ng/ml) of sulcardine free base in blood plasma.

As used herein, “AUC” refers to area under the plasma concentration versus time curve for sulcardine free base.

All sulcardine dosages recorded herein are reported in terms of sulcardine sulfate, unless otherwise specified.

All blood plasma levels of sulcardine recorded herein are reported in terms of the free sulcardine base, which is the active moiety responsible for the ion channel activity of sulcardine.

As used herein and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

“Treat,” “treatment,” and “treating” are employed in this description to refer to administering a pharmaceutical composition or formulation for prophylactic and/or therapeutic purposes. The term “therapeutic treatment” refers to administering treatment to a patient already suffering from a condition such as arrhythmia. Thus, in preferred embodiments, treating is the administration to a mammal of therapeutically effective amounts of an antiarrhythmic agent.

A “subject” of treatment is a human or non-human mammal. Non-human mammals include, for example, a simian, a murine, a canine, a leporid, such as a rabbit, livestock, sport animals, and pets. As used herein and unless otherwise specified, a “patient” is a human subject.

A “treatment population” refers to a group of clinically typical patients receiving the treatment and a typical response that would be expected from said patents.

An “antiarrhythmic agent,” as used herein, refers to a molecule having a therapeutic effect of treating arrhythmia or alleviating associated symptoms in a subject. Non-limiting examples of arrhythmias include supraventricular tachyarrhythmia such as atrial fibrillation, atrial flutter, premature ventricular contractions, ventricular tachycardia, and ventricular fibrillation. In one aspect, an antiarrhythmic agent is sulcardine, or a pharmaceutically acceptable salt thereof. In another aspect, an antiarrhythmic agent is sulcardine sulfate.

As used herein, a pharmaceutically acceptable salt of sulcardine can be the active agent in a formulation useful for treating arrhythmia. Illustrative of such sulcardine salts are: (A) inorganic acid salts such as acetate, borate, bicarbonate, sulfate, hydrochloride, bromides, chlorides, iodide, hydrobromide, hydroiodide, nitrate, phosphate, diphosphate, and fluorophosphate salts; (B) organic acid salts such as amsonate (4,4-diaminostilbene-2,2-disulfonate), bitartrate, butyrate, citrate, calcium edetate, camsylate, edisylate, estolate, esylate, glutamate, gluconate, gluceptate, lactate, lactobionate, laurate, malate, maleate, mandelate, methylbromide, methylnitrate, methylsulfate, mucate, oleate, oxalate, palmitate, pamoate (1,1-methene-bis-2-hydroxy-3-naphthoate, cinbonate), pamoate, pantothenate, salicylate, stearate, subacetate, succinate, sulfate, sulfosalicylate, suramate, propionate, valerate, fiunarate, fumarate, and tartrate salts; and (C) alkali metal salts and alkali earth salts, such as the sodium, potassium, lithium and calcium salts of sulcardine. In this context, a pharmaceutically acceptable salt can have more than one charged atom in its structure and, hence, one or more counterions.

The phrases “effective amount,” “therapeutically effective amount,” and “pharmaceutically effective amount” denote an amount of an active agent, such as an antiarrhythmic agent as presently disclosed, that has a therapeutic effect. The doses of the active agent which are useful in treatment are therapeutically effective amounts. Thus, a therapeutically effective amount is an amount of the active agent that produces the desired therapeutic effect, as judged by clinical trial results and/or model animal studies. In particular embodiments, the active agent is administered in a pre-determined dose; hence, a therapeutically effective amount would be an amount of the dose administered. This amount also can depend upon the patient's height, weight, sex, age and medical history.

A “carrier” or “excipient” is a compound or material used to facilitate administration of the compound, for example, to control the release and/or bioavailability of the compound. Solid carriers include, e.g., starch, lactose, dicalcium phosphate, sucrose, and kaolin. Liquid carriers include, e.g., sterile water, saline, buffers, non-ionic surfactants, and edible oils such as oil, peanut and sesame oils. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

The phrases “pharmaceutically acceptable carrier” and “pharmaceutically acceptable excipient” can note any and all solvents, dispersion media, coatings, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. Suitable pharmaceutically acceptable excipients include, but are not limited to, buffers, diluents, tonicity agents, stabilizers, antioxidants, preservatives and mixtures thereof.

The term “buffer” denotes a pharmaceutically acceptable excipient, which stabilizes the pH of a pharmaceutical preparation. Suitable buffers are known in the art and can be found in the literature. Pharmaceutically acceptable buffers comprise but are not limited to glycine-buffers, histidine-buffers, citrate-buffers, succinate-buffers and phosphate-buffers. Independently from the buffer used, the pH can be adjusted at a value from about 2 to about 9, or alternatively from about 2.5 to about 7, or alternatively from about 3 to about 5 or alternatively about 3 with an acid or a base known in the art, e.g., succinic acid, hydrochloric acid, acetic acid, phosphoric acid, sulfuric acid and citric acid, sodium hydroxide and potassium hydroxide.

Suitable buffers include, without limitation, glycine buffer, histidine buffer, 2-morpholinocthanesulfonic acid (MES), cacodylate, phosphate, acetate, succinate, and citrate. In one aspect, the buffer is a glycine buffer. In another aspect, the buffer is a histidine buffer. The concentration of the buffer can be between about 1 mM and about 100 mM, or alternatively about 2 mM to about 40 mM, or alternatively about 5 mM to about 20 mM.

The term “average” as used herein, refers to a value that is an average of values derived from a variable number of patients and/or subjects given the same treatment and/or compound.

The term “mean average” as used herein, refers to a value that is the sum of the values from the variable population of patients divided by the number of patients in the population given the same treatment and/or compound.

### Method of Use

Pharmaceutical compositions described herein can be used to protect against malignant prolongation of the QT interval of a subject in need of such protection. Several potential therapeutic agents are known to prolong the QT interval. Without being bound by theory, it is believed that many of these drugs prolong the QT interval by delaying repolarization of ventricular myocytes, e.g., by inhibiting potassium rectifier channels responsible for repolarizing cells and maintaining polarization in Phases 3 and 4 of the contraction/relaxation cycle. The pharmaceutical compositions described herein protect against malignant QT interval prolongation by shortening the J-Tp interval—i.e., the interval from the end of the QRS complex (J-point) to the apex of the T peak (Tp). JTp interval reduction is the 12-lead ECG manifestation of a decrease in duration in the early repolarization period, which is the cellular mechanism underlying the protective mechanism of sulcardine described herein. Without being bound by theory, it is believed that the compositions described herein shorten the J-Tpeak interval (JTp) by inhibiting one or more of the late-opening sodium channels, thereby reducing late sodium current (I_{Na.late}), and/or long-lasting (L-type) calcium channels, thereby reducing L-type calcium current (I_{Ca.L}), with the results of shortening early repolarization, which is represented in the ECG as the J-Tp interval.

The major charge-carrying ions in cardiomyocytes are Na⁺, K⁺, Ca²⁺, and Cl—. A typical cardiac (contraction-relaxation) cycle is caused by coordinated transport of these ions across the myocyte cell membrane. At rest, there is a negative charge across the cell membrane. Contraction commences when the cell is stimulated from without, which results in rapid depolarization, due to rapid positive ion flow into the cell. Relaxation progresses through various phases caused by rebalancing of positive ions across the cell membrane.

The change in voltage across the cell membrane over time is referred to as the action potential. The action potential of a cardiac myocyte has 5 phases. In phase 0, the rapid depolarization stage, the action potential changed from negative to positive. In phase 1, the early rapid repolarization phase, repolarization begins. Phase 2, plateau, is a period during which inward and outward positive ion flows are approximately balanced. In phase 3, final repolarization, positive ions flow out of the cell. In phase 4, resting membrane potential, inward and outward flows are kept in balance to maintain constant negative voltage.

In typical atrial and ventricular myocytes, phase 0 is caused by the opening of fast Na⁺ channels, phase 1 is caused by several K⁺ channels, phase 2 is caused by the activity of Ca²⁺ channels and some Na⁺ channels balanced with K⁺ channels, and phase 3 is caused by K⁺ channels, which are different from those active in phase 1. The absolute and relative prevalences and activities of these channels differ in atrial and ventricular myocytes and in normal versus diseased tissue (e.g., tachycardia-remodeled myocardium).

In practice, the QT interval is generally corrected for heart rate. The corrected QT interval, QTc, reflects such correction. Various corrections are known. One of these corrections employs Fridericia's formula (QTcF=(QT/(RR)^{1/3}) to correct for changes in QT due to changes in heart rate, wherein QT is the duration in milliseconds of the Q-T interval and RR is the duration of the interval from one R peak to the next. Although RR is recorded in ms, the value is treated as dimensionless, so the value of QTcF is reported in milliseconds. Although other corrections are known in the art, as used herein, unless otherwise specified, QTc refers to QTcF, whereas QT refers to uncorrected QT interval values.

JTp is often corrected for changes in the interval due to heart rate changes. The corrected interval, JTpc, reflects such correction. Various corrections are known. One of these corrections employs Fridericia's formula (JTpcF=(JTp/(RR)^{1/3}) to correct for changes in JTp due to changes in heart rate, wherein JTp is the duration in milliseconds of the interval from the J-point to the peak of the T wave, and RR is the duration of the interval from one R peak to the next. Although RR is recorded in ms, the value is treated as dimensionless, so the value of JTpcF is reported in milliseconds. Although other corrections are known in the art, as used herein, unless otherwise specified, JTpc refers to JTpcF, whereas JTp refers to the uncorrected JTp interval value.

The inventors have surprisingly found that JTp decreases sharply as sulcardine dose increases. This relationship holds whether the dose is measured in terms of sulcardine administered to the patient or in terms of exposure—Cmax or AUC. This shortening of the JTp interval, which holds whether JTp is corrected (JTpc) or uncorrected, is robust across a wide range of dosages and exposure levels. This shortening effect protects acute AF patients treated with sulcardine from QT interval prolongation, thereby protecting the patients from pathological cardiac arrhythmias, such as ventricular fibrillation, that are often associated with treatment with antiarrhythmic agents.

The active agent in HBI-3000 (sulcardine sulfate) is the free base of sulcardine. While HBI-3000 is the salt form of sulcardine referenced in the following description, one skilled in the art will recognize that any bioavailable form of sulcardine, e.g. sulcardine edisylate, can be used in an analogous manner, e.g. by intravenous infusion, oral administration, etc., taking into account the differences in molecular weight between salt species. Unless otherwise specified herein, dosages are reported in milligrams (mg) of sulcardine sulfate. As the moiety responsible for the ion channel activity of sulcardine is the free base of sulcardine, one of skill in the art will understand that if a different salt of sulcardine were employed, the dosage could be adjusted to provide an equivalent dose of sulcardine free base. Other adjustments may also be made to compensate for differences in solubility and bioavailability.

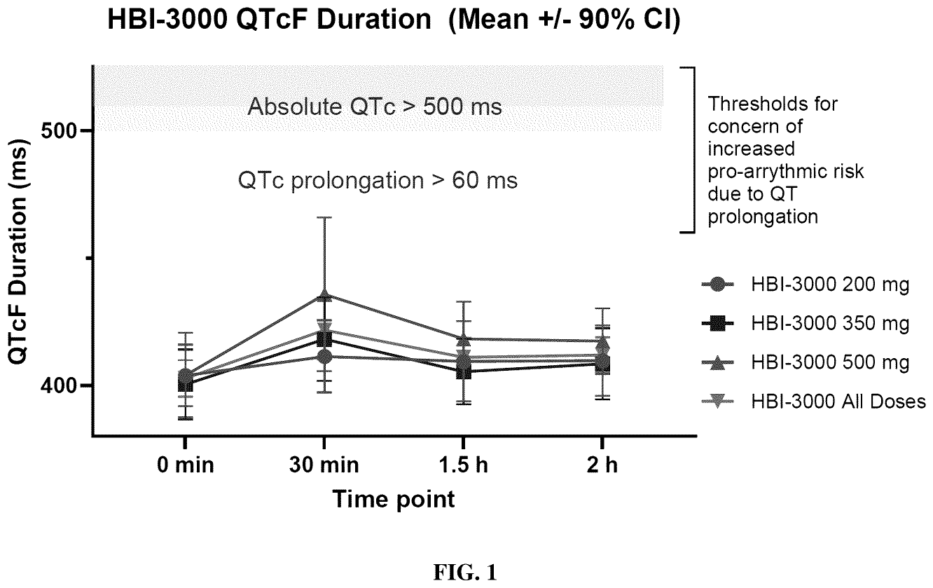

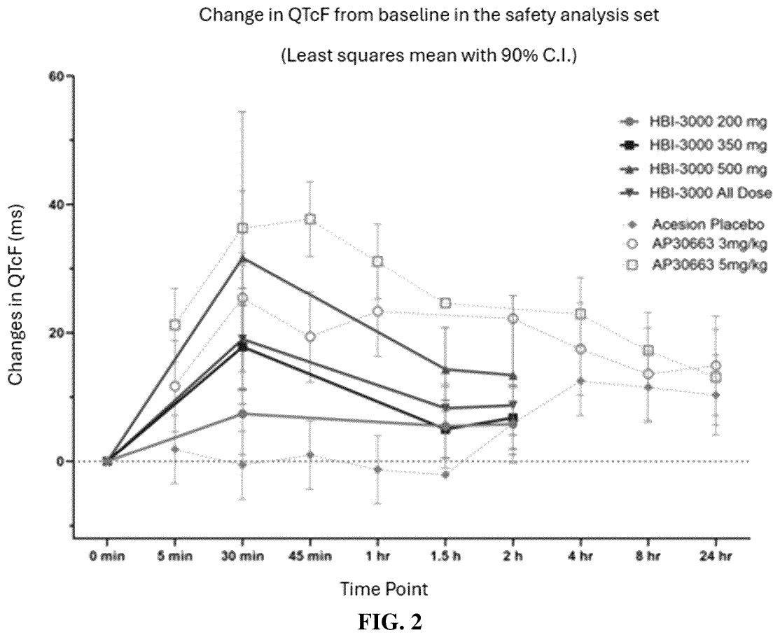

The inventors have shown that sulcardine protects against pathological prolongation of the QT interval. FIG. 1 shows the results of administering doses of 200 mg, 350 mg, and 500 mg of sulcardine as HBI-3000 (sulcardine sulfate), equivalent to 165 mg, 289 mg, and 413 mg of sulcardine free base, respectively. Since the active form of the drug is the free base sulcardine, and the free base dissociates from the salt form in the blood, similar results are expected from alternative salt forms and regardless of how the drug is administered (IV, oral, etc.). QTcF duration (ms) is shown on the y-axis at time points of 0 and 30 minutes and 1.5 and 2 hours after administration begins. Beginning QTcF duration was about 400 ms for each arm of the trial and QTc prolongation of greater than 60 ms were considered potentially pathogenic. There was no prolongation in mean QTc of greater than 60 ms, and no induced ventricular fibrillation, observed in any of the study arms. FIG. 2 compares the performance of HBI-3000 compared to the antiarrhythmic drug from Acesion Pharma, AP30663.

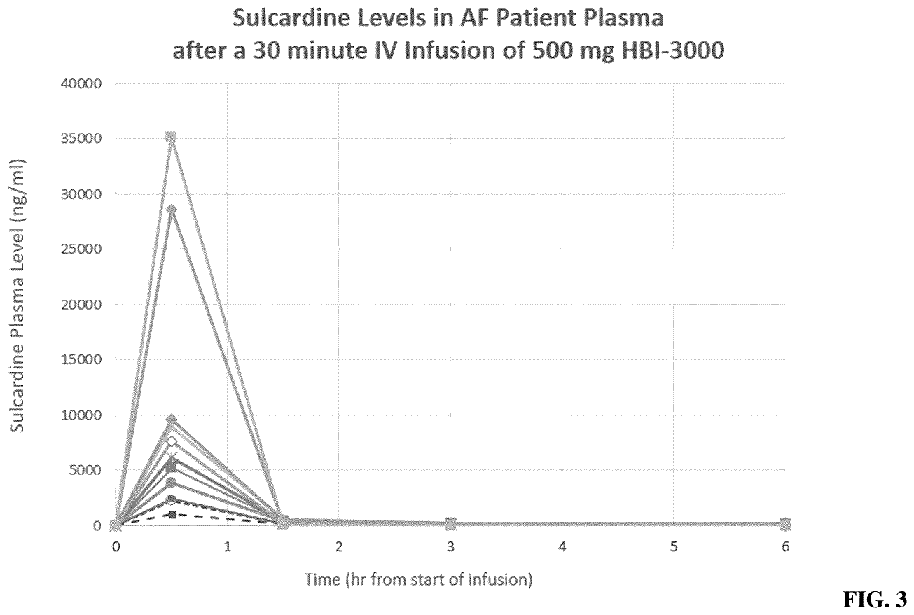

The inventors have discovered that, when sulcardine is administered to human subjects in sinus rhythm or in arrhythmia (AF), maximum plasma concentration of sulcardine (Cmax) and total plasma exposure of sulcardine (AUC) can vary greatly from subject to subject. See, for example, single patient data illustrated in FIG. 3. Patients (subjects) were administered 500 mg HBI-3000 over 30 minutes by IV infusion. The single patient Cmax values varied from well below 5,000 ng/ml to approximately 35,000 ng/ml. Despite this widely varying sulcardine exposure, no patient experienced pathological QT prolongation. While not wishing to be bound by theory, the inventors theorize that sulcardine protects against QT prolongation by shortening the J-Tpeak (JTp) interval, thereby compensating for QT prolongation that would be expected based on the activity of sulcardine on other repolarizing cation channels. Indeed, compensatory JTp shortening is dose- and exposure-dependent, meaning that the QT protective effect is durable across large ranges of sulcardine dosage, Cmax, and AUC.

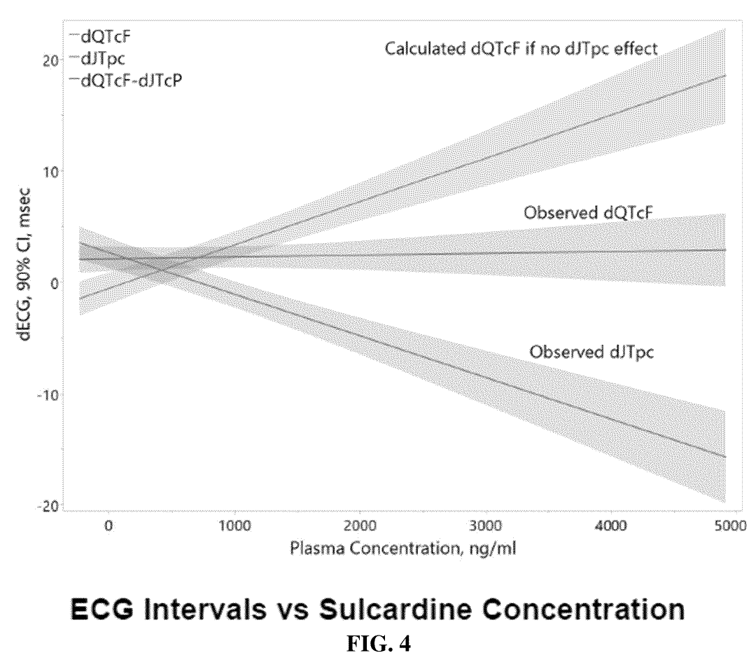

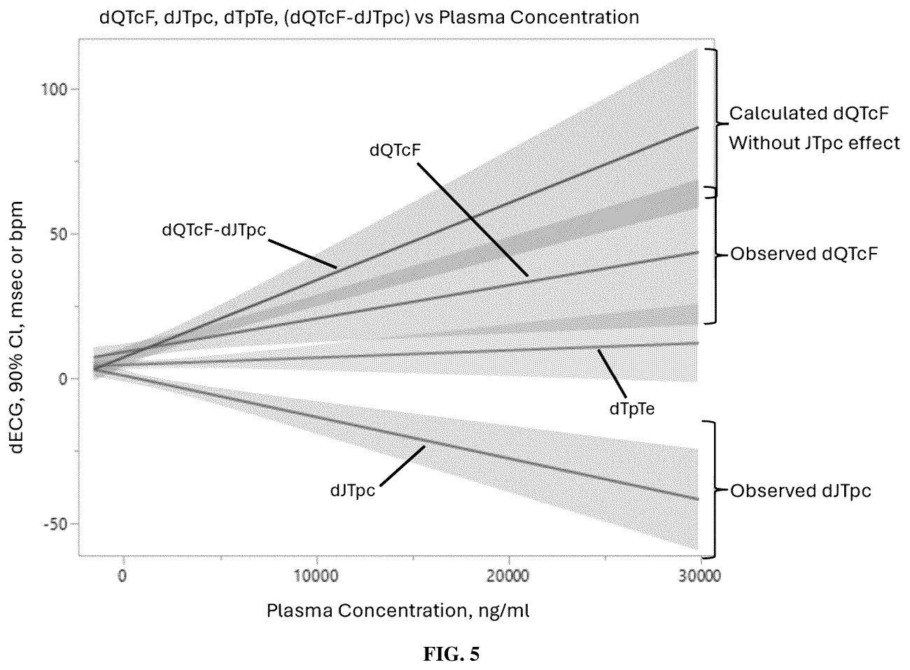

FIG. 4 and FIG. 5 show linear regression plots of observed and calculated duration effects on JTp (JTpc), QT (QTcF), and QT if there were no JTp shortening (dJTpc). As can be seen in FIG. 4, dJTpc decreases (negative) over a range of 0 to 5,000 ng/ml of plasma sulcardine. As can be seen in FIG. 5, this trend holds over a range of 0 to 30,000 ng/ml of plasma sulcardine. This linear decrease in JTp offsets the expected increase in QT, as the slope of the observed dQTcF is lower than the slope of the calculated (expected) dQTcF curve over the same ranges of values. While not wishing to be bound by theory, the inventors believe that the observed dQTcF is lower than the expected dQTcF over a range of values of up to about 35,000 ng/ml (or higher) due to the compensating shortening of JTp (i.e., negative JTpc curve).

Thus, some embodiments described herein provide a method of treating atrial fibrillation (AF) or atrial flutter without causing pathological prolongation of the QT interval over dosage ranges of sulcardine that cause sulcardine Cmax to be from about 100 to about 35,000 ng/ml of sulcardine free base. In some embodiments, pathological QT interval prolongation (dQTc) is greater than about 60 ms. Thus, some embodiments provide a method of treating atrial fibrillation or atrial flutter, comprising administering a dosage of sulcardine, or a pharmaceutically acceptable salt thereof, that causes a sulcardine Cmax to be from about 100 to about 35,000 ng/ml and the dQTc to be from 0 to 60 ms. In some embodiments, dQTc is from 0 to 50 ms. In some embodiments dQTc is from 0 to 40 msec. Suitable sulcardine doses include 200 mg to 500 mg of sulcardine sulfate (equivalent to 165 mg to 413 mg sulcardine free base) or a molar equivalent of a suitable salt of sulcardine, such as sulcardine sulfate or sulcardine edisylate. In some embodiments, the sulcardine, or pharmaceutically acceptable salt, is administered as an intravenous infusion over a period of from 15 min. to 60 min., e.g., about 30 min.

In some embodiments, QT interval protective compounds can be used in the treatment of one or more arrhythmias, such as atrial fibrillation (AF), atrial flutter, ventricular fibrillation (VF), etc. In some embodiments, QT interval protective compounds can be used to protect against inducement of one or more types of fibrillation, such as AF, VF, and TdP. In some embodiments, QT interval protective compounds can be used to shorten the JTp interval, thereby shortening the QT interval relative to what would have been expected from the compounds' I_{Kr} inhibiting activity. In some embodiments, the QT interval protective compound is, or comprises, sulcardine or a pharmaceutically acceptable salt thereof, such as sulcardine sulfate or sulcardine edisylate.

In some embodiments, the pharmaceutical compositions described herein are administered in an amount that causes QT prolongation (dQT) of less than a pre-selected threshold. The pre-selected threshold may be selected such that, within the confidence interval of the testing method, dQT is not likely to cause fibrillation, such as AF or VF. In some embodiments, the threshold is no more than 60 ms, less than 60 ms, no more than 50 ms, less than 50 ms, no more than 40 ms, or less than 40 ms. In some embodiments, QT is QTcF.

In some embodiments, the QT protective compound is sulcardine, or a pharmaceutically acceptable salt thereof, such as sulcardine sulfate or sulcardine mono-edisylate. Sulcardine demonstrates a linear decrease in JTp interval that appears to compensate for expected increase in QT interval over a broad range of blood plasma concentrations, thereby resulting in an observed QT interval that is less than the expected QT interval (QTe). See FIG. 5. The QTc interval has been observed to remain below a potentially malignant threshold in subjects administered an amount of sulcardine sulfate that causes a plasma concentration of sulcardine of up to 30,000 ng/mL. Thus, in some embodiments of the methods described herein, the sulcardine compositions are administered in an amount that causes a peak blood plasma concentration of sulcardine of less than or equal to 30,000 ng/ml. In some embodiments, the threshold is no more than 60 ms, less than 60 ms, no more than 50 ms, less than 50 ms, no more than 40 ms, or less than 40 ms.

Some embodiments provide a method of treating atrial fibrillation (AF) or atrial flutter, comprising administering to a human subject a dose of sulcardine, wherein administering the dose of sulcardine to the human subject causes a sulcardine Cmax in the human subject of about 100 ng/ml to about 35,000 ng/ml and a QTc prolongation of 0 ms to 60 ms. In some embodiments, the QTc prolongation is less than 50 ms. In some embodiments the QTc prolongation is 40 ms. In some embodiments the QTc is less than about 500 ms.

Some embodiments provide a method of protecting against QTc prolongation in a human subject, comprising administering to the human subject an effective amount of a compound, wherein the compound has a JTpc interval shortening activity and the human subject has an electrocardiogram (ECG) having a QTc. In some embodiments, the JTpc interval shortening activity comprises inhibiting, in a cardiomyocyte, an inward sodium current, an inward calcium current, or both. In some embodiments, the inward sodium current comprises both an inward early sodium current and a late inward sodium current. In some embodiments, administering the compound to the human subject causes the QTc prolongation to be less than about 60 ms. In some embodiments, the QTc is less than about 500 ms.

Some embodiments provide a method of protecting against QTc prolongation in a human subject, comprising administering to the human subject an effective amount of a compound, wherein the compound inhibits one or both of a late sodium inward ion current and an L-type calcium current in cardiomyocytes, thereby shortening a JTpc interval. In some embodiments, the compound inhibits both the late sodium inward ion current and the L-type calcium current in cardiomyocytes. In some embodiments, the QTc prolongation is less than about 60 ms, less than 50 ms, or less than 40 ms. In some embodiments, the human subject has a QTc of less than about 500 ms. In some embodiments, the compound is sulcardine or a pharmaceutically acceptable salt of sulcardine. In some embodiments, the effective amount of sulcardine, or a pharmaceutically acceptable salt of sulcardine, is about 200 mg to about 500 mg sulcardine sulfate, equivalent to 165 mg to about 413 mg free base of sulcardine. In some embodiments, the effective amount of the compound causes a maximum blood plasma concentration (Cmax) of up to 5,000 ng/ml of the free base of sulcardine.

Some embodiments described herein provide a pharmaceutical composition comprising a compound that protects against QT interval prolongation when administered to a subject. In some embodiments, the composition protects against QT prolongation by producing a blood plasma concentration-dependent shortening of the J-Tp interval (JTp). In some embodiments, JTp is JTpc, i.e., JTp interval corrected for changes in heart rate (HR). In some embodiments, QT is QTc, i.e., QT interval corrected for changes in heart rate (HR). In some embodiments, QTc is QTcF. In some embodiments, the pharmaceutical composition produces no change or a slight increase in TpTe. In some embodiments, QT prolongation is less than would have been expected based on the I_{Kr} inhibiting activity of the compound. In some embodiments, the compound that protects against QT interval prolongation is sulcardine, or a pharmaceutically acceptable salt thereof, such as sulcardine sulfate or sulcardine edisylate.

Since QT prolongation with sulcardine tends to be linear in a blood plasma concentration of up to at least 30,000 ng/ml of sulcardine, it is expected that maximum QT prolongation will occur when administration of sulcardine, or a pharmaceutically acceptable salt thereof, causes the maximum blood plasma concentration (Cmax) of sulcardine. The time from administration of sulcardine, or a pharmaceutically acceptable salt thereof, to Cmax is referred to as Tmax. Accordingly, the maximum tolerated maximum dose of sulcardine, or a pharmaceutically acceptable salt thereof, will depend on the slope of the blood plasma concentration curve (dC/dT). For routes of administration that cause a steep concentration per unit time curve (high dC/dT), Cmax will be reached more quickly (short Tmax) and thus lower dosages will result in dQTc within the desired range of ≤60 ms, ≤50 ms, or <40 ms and/or an absolute QTc value of less than 500 ms. For intravenous administration for a period of ˜ 30 min., the administered dose of sulcardine, or a pharmaceutically acceptable salt thereof, should be in a range of about 200 to 500 sulcardine sulfate (165 to 413 mg free base sulcardine), preferably about 200 to 450 (165 to 372 mg free base sulcardine), more preferably about 200 to 400 (165 to 331 mg free base sulcardine), e.g., about 350 mg of sulcardine (289 mg free base sulcardine; or a molar equivalent amount of a salt of sulcardine).

For other routes of administration, the dose of sulcardine, or pharmaceutically acceptable salt thereof, may be adjusted upward so long as the observed dC/dT of sulcardine by the route of administration is less than or equal to the dC/dT observed for intravenous administration over ˜ 30 min. In other words, the sulcardine dose may be adjusted upward from the intravenous dose to a limit established by the maximum dosage that causes a Cmax in the patient of ≤30,000 ng/mL of sulcardine.

In some embodiments, the pharmaceutically acceptable salt in the composition is sulcardine sulfate or sulcardine edisylate.

In some embodiments, the pharmaceutical composition provided herein further comprises a pharmaceutically acceptable excipient, such as one or more of: water for injection, an osmolality-balancing agent, a buffer, a preservative, or other pharmaceutically acceptable excipient.

In some embodiments, Tmax is at about 30 minutes.

In some embodiments, sulcardine, or a pharmaceutically acceptable salt thereof, is administered at a dose of from about 100 mg to about 600 mg of sulcardine (sulcardine sulfate). In some embodiments, sulcardine, or a pharmaceutically acceptable salt thereof, is administered at a dose of from about 150 mg to about 600 mg of sulcardine (sulcardine sulfate). In some embodiments, sulcardine, or a pharmaceutically acceptable salt thereof, is administered at a dose of from about 200 mg to about 600 mg of sulcardine (sulcardine sulfate). In some embodiments, sulcardine, or a pharmaceutically acceptable salt thereof, is administered at a dose of from about 200 mg to about 500 mg of sulcardine (sulcardine sulfate). In some embodiments, sulcardine, or a pharmaceutically acceptable salt thereof, is administered at a dose of about 350 mg of sulcardine (sulcardine sulfate).

In some embodiments, sulcardine, or a pharmaceutically acceptable salt thereof, is administered at a dose of from about 100 mg to about 450 mg of sulcardine (sulcardine sulfate). In some embodiments, sulcardine, or a pharmaceutically acceptable salt thereof, is administered at a dose of about 150 mg to about 450 mg of sulcardine (sulcardine sulfate). In some embodiments, sulcardine, or a pharmaceutically acceptable salt thereof, is administered at a dose of from about 200 mg to about 450 mg of sulcardine (sulcardine sulfate). In some embodiments, sulcardine, or a pharmaceutically acceptable salt thereof, is administered at a dose of from about 200 mg to about 400 mg of sulcardine (sulcardine sulfate). In some embodiments, sulcardine, or a pharmaceutically acceptable salt thereof, is administered at a dose of about 350 mg of sulcardine (sulcardine sulfate).

In some embodiments, sulcardine, or a pharmaceutically acceptable salt thereof, is administered at a dose of from about 100 mg to about 600 mg of sulcardine (free base). In some embodiments, sulcardine, or a pharmaceutically acceptable salt thereof, is administered at a dose of from about 150 mg to about 600 mg of sulcardine (free base). In some embodiments, sulcardine, or a pharmaceutically acceptable salt thereof, is administered at a dose of from about 200 mg to about 600 mg of sulcardine (free base). In some embodiments, sulcardine, or a pharmaceutically acceptable salt thereof, is administered at a dose of from about 200 mg to about 500 mg of sulcardine (free base). In some embodiments, sulcardine, or a pharmaceutically acceptable salt thereof, is administered at a dose of about 350 mg of sulcardine (free base).

In some embodiments, sulcardine, or a pharmaceutically acceptable salt thereof, is administered at a dose of from about 100 mg to about 450 mg of sulcardine (free base). In some embodiments, sulcardine, or a pharmaceutically acceptable salt thereof, is administered at a dose of about 150 mg to about 450 mg of sulcardine (free base). In some embodiments, sulcardine, or a pharmaceutically acceptable salt thereof, is administered at a dose of from about 200 mg to about 450 mg of sulcardine (free base). In some embodiments, sulcardine, or a pharmaceutically acceptable salt thereof, is administered at a dose of from about 200 mg to about 400 mg of sulcardine (free base). In some embodiments, sulcardine, or a pharmaceutically acceptable salt thereof, is administered at a dose of about 350 mg of sulcardine (free base).

In some embodiments, the dosages of sulcardine may also include about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, and dosage in between. The dosages may also include about 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, and dosages within ranges between any of the preceding dosages, wherein all dosages are expressed in terms of sulcardine sulfate.

In some embodiments, sulcardine or a pharmaceutically acceptable salt thereof is administered. In some embodiments, sulcardine sulfate is administered. In some embodiments, sulcardine or a pharmaceutically acceptable salt thereof is administered by parenteral administration. In some embodiments, parenteral administration is intravenous infusion, or intramuscular or subcutaneous injection. In some embodiments, sulcardine or a pharmaceutically acceptable salt thereof is administered by intravenous infusion. In some embodiments, sulcardine or a pharmaceutically acceptable salt thereof is administered by intramuscular injection. In some embodiments, sulcardine or a pharmaceutically acceptable salt thereof is administered orally.

In some embodiments, sulcardine, or a pharmaceutically acceptable salt thereof, is administered as a solution with a concentration of about 50 mg/mL. In some embodiments, the solution is diluted to about 8 mg/ml or less to deliver about 200-500 mg dose of sulcardine sulfate in a volume of 50 ml to a patient.

In some embodiments, sulcardine, or a pharmaceutically acceptable salt thereof, is administered over a period of from about 15 minutes to about 2 hours. In some embodiments, sulcardine, or a pharmaceutically acceptable salt thereof, is administered over a period of from about 30 minutes to about 1 hour. In some embodiments, sulcardine, or a pharmaceutically acceptable salt thereof, is administered over a period of less than about 1 hour. In some embodiments, sulcardine, or a pharmaceutically acceptable salt thereof, is administered over a period of about 30 minutes. In some embodiments, sulcardine, or a pharmaceutically acceptable salt thereof, is administered over a period of about 15 minutes.

In some embodiments, sulcardine or a pharmaceutically acceptable salt thereof, is administered at a rate that produces a normal sinus rhythm without producing an arrhythmia or a clinically significant change in heart rate of blood pressure. In some embodiments, sulcardine or a pharmaceutically acceptable salt thereof, is administered at a rate that does not produce an arrhythmia or a clinically significant change in heart rate of blood pressure. In some embodiment, the method increases or decreases diastolic and/or systolic blood pressure by no more than 25%. In some embodiments, the method does not induce a 2nd or 3rd degree heart block.

In some embodiments, sulcardine or a pharmaceutically acceptable salt thereof, is administered from about after the onset of symptoms. In some embodiments, sulcardine or a pharmaceutically acceptable salt thereof, is administered from about 15 minutes to about 72 hours after the onset of symptoms. In some embodiments, sulcardine or a pharmaceutically acceptable salt thereof, is administered no more than about 72 hours after the onset of symptoms. In some embodiments, sulcardine, or a salt thereof, is administered without need for anti-coagulation. In some embodiments, sulcardine or a pharmaceutically acceptable salt thereof, is administered after less than 7 days after the onset of symptoms without need for anticoagulation. In some embodiments, sulcardine or a pharmaceutically acceptable salt thereof, is administered after about 72 hours after the onset of symptoms and after anti-coagulation therapy.

In the above embodiments, the ECG parameter changes may be dependent on the level of average maximum plasma concentration (Cmax) of sulcardine or a pharmaceutically acceptable salt thereof in a patient. In some embodiments, the average maximum plasma concentration (Cmax) of sulcardine or a pharmaceutically acceptable salt thereof in a given patient is about 1,000 ng/mL to about 10,000 ng/mL. In some embodiments, the level of average maximum plasma concentration (Cmax) of sulcardine or a pharmaceutically acceptable salt thereof in a given patient is about 1,000 ng/mL to about 2,000 ng/ml, about 1,000 ng/ml to about 3,000 ng/mL, about 1,000 ng/mL to about 4,000 ng/mL, about 1,000 ng/mL to about 5,000 ng/mL, about 1,000 ng/mL to about 6,000 ng/mL, about 1,000 ng/ml to about 7,000 ng/ml, about 1,000 ng/ml to about 8,000 ng/mL, about 1,000 ng/ml to about 9,000 ng/ml, about 1,000 ng/ml to about 10,000 ng/mL, about 2,000 ng/ml to about 3,000 ng/ml, about 2,000 ng/ml to about 4,000 ng/mL, about 2,000 ng/mL to about 5,000 ng/mL, about 2,000 ng/mL to about 6,000 ng/mL, about 2,000 ng/mL to about 7,000 ng/mL, about 2,000 ng/ml to about 8,000 ng/ml, about 2,000 ng/mL to about 9,000 ng/mL, about 2,000 ng/mL to about 10,000 ng/mL, about 3,000 ng/ml to about 4,000 ng/mL, about 3,000 ng/mL to about 5,000 ng/mL, about 3,000 ng/ml to about 6,000 ng/ml, about 3,000 ng/ml to about 7,000 ng/ml, about 3,000 ng/ml to about 8,000 ng/ml, about 3,000 ng/mL to about 9,000 ng/mL, about 3,000 ng/mL to about 10,000 ng/mL, about 4,000 ng/mL to about 5,000 ng/mL, about 4,000 ng/mL to about 6,000 ng/ml, about 4,000 ng/mL to about 7,000 ng/mL, about 4,000 ng/mL to about 8,000 ng/ml, about 4,000 ng/ml to about 9,000 ng/mL, about 4,000 ng/ml to about 10,000 ng/ml, about 5,000 ng/mL to about 6,000 ng/mL, about 5,000 ng/ml to about 7,000 ng/mL, about 5,000 ng/mL to about 8,000 ng/mL, about 5,000 ng/mL to about 9,000 ng/mL, about 5,000 ng/ml to about 10,000 ng/mL, about 6,000 ng/mL to about 7,000 ng/ml, about 6,000 ng/ml to about 8,000 ng/mL, about 6,000 ng/mL to about 9,000 ng/mL, about 6,000 ng/ml to about 10,000 ng/mL, about 7,000 ng/ml to about 8,000 ng/mL, about 7,000 ng/ml to about 9,000 ng/ml, about 7,000 ng/ml to about 10,000 ng/mL, about 8,000 ng/ml to about 9,000 ng/mL, about 8,000 ng/ml to about 10,000 ng/mL, or about 9,000 ng/mL to about 10,000 ng/mL. In some embodiments, the level of average maximum plasma concentration (Cmax) of sulcardine or a pharmaceutically acceptable salt thereof in a given patient is about 1,000 ng/ml, about 2,000 ng/mL, about 3,000 ng/mL, about 4,000 ng/mL, about 5,000 ng/ml, about 6,000 ng/mL, about 7,000 ng/mL, about 8,000 ng/mL, about 9,000 ng/mL, or about 10,000 ng/ml. In some embodiments, the level of average maximum plasma concentration (Cmax) of sulcardine or a pharmaceutically acceptable salt thereof in a given patient is at least about 1,000 ng/mL, about 2,000 ng/mL, about 3,000 ng/mL, about 4,000 ng/mL, about 5,000 ng/mL, about 6,000 ng/ml, about 7,000 ng/mL, about 8,000 ng/mL, or about 9,000 ng/mL. In some embodiments, the level of average maximum plasma concentration (Cmax) of sulcardine or a pharmaceutically acceptable salt thereof in a given patient is at most about 2,000 ng/ml, about 3,000 ng/ml, about 4,000 ng/mL, about 5,000 ng/mL, about 6,000 ng/ml, about 7,000 ng/ml, about 8,000 ng/ml, about 9,000 ng/ml, or about 10,000 ng/mL.

In some embodiments, the average maximum plasma concentration (Cmax) of sulcardine or a pharmaceutically acceptable salt thereof in a given patient is about 1,000 ng/ml to about 30,000 ng/mL. In some embodiments, the level of average maximum plasma concentration (Cmax) of sulcardine or a pharmaceutically acceptable salt thereof in a given patient is about 1,000 ng/ml to about 25,000 ng/ml, about 1,000 ng/mL to about 20,000 ng/ml, about 1,000 ng/mL to about 15,000 ng/mL, about 1,000 ng/mL to about 10,000 ng/ml, about 1,000 ng/ml to about 7,500 ng/ml, about 1,000 ng/mL to about 5,000 ng/mL, about 1,500 ng/mL to about 30,000 ng/ml, about 25,000 ng/mL to about 20,000 ng/ml, about 1,500 ng/ml to about 15,000 ng/mL, about 7,500 ng/mL to about 5,000 ng/ml, about 2,000 ng/mL to about 30,000 ng/mL, about 2,000 ng/mL to about 25,000 ng/mL, about 2,000 ng/mL to about 20,000 ng/mL, about 2,000 ng/ml to about 15,000 ng/ml, about 2,000 ng/mL to about 10,000 ng/ml, about 2,000 ng/ml to about 7,500 ng/mL, about 2,000 ng/mL to about 5,000 ng/ml, about 5,000 ng/ml to about 30,000 ng/mL, about 5,000 ng/ml to about 25,000 ng/ml, about 5,000 ng/ml to about 20,000 ng/ml, about 5,000 ng/mL to about 15,000 ng/mL, about 5,000 ng/ml to about 12,500 ng/ml, or about 5,000 ng/mL to about 10,000 ng/mL. In some embodiments, the level of average maximum plasma concentration (Cmax) of sulcardine or a pharmaceutically acceptable salt thereof in a given patient is about 1,000 ng/mL, about 2,000 ng/mL, about 6,000 ng/ml, about 7,500 ng/mL, about 10,000 ng/ml, about 12,500 ng/mL, about 15,000 ng/ml, about 17,500 ng/mL, about 20,000 ng/mL, about 22,500 ng/mL, about 25,000 ng/mL, about 30,000 ng/mL, about 35,000 ng/ml, or more.

In the above embodiments, the ECG parameter changes may be dependent on the level of average plasma concentration and/or average plasma exposure (AUC) of sulcardine or a pharmaceutically acceptable salt thereof in a patient. In some embodiments, the level of average plasma concentration and/or average plasma exposure (AUC) of sulcardine or a pharmaceutically acceptable salt thereof in a patient is about 1,000 ng·h/mL to about 10,000 ng·h/mL. In some embodiments, the level of average plasma concentration and/or average plasma exposure (AUC) of sulcardine or a pharmaceutically acceptable salt thereof in a given patient is about 1,000 ng·h/mL to about 2,000 ng·h/mL, about 1,000 ng·h/mL to about 3,000 ng·h/mL, about 1,000 ng·h/mL to about 4,000 ng·h/mL, about 1,000 ng·h/mL to about 5,000 ng·h/mL, about 1,000 ng·h/mL to about 6,000 ng·h/mL, about 1,000 ng·h/mL to about 7,000 ng·h/mL, about 1,000 ng-h/mL to about 8,000 ng·h/mL, about 1,000 ng-h/mL to about 9,000 ng·h/mL, about 1,000 ng·h/mL to about 10,000 ng·h/mL, about 2,000 ng·h/mL to about 3,000 ng·h/mL, about 2,000 ng·h/mL to about 4,000 ng·h/mL, about 2,000 ng·h/mL to about 5,000 ng·h/mL, about 2,000 ng·h/mL to about 6,000 ng·h/mL, about 2,000 ng·h/mL to about 7,000 ng·h/mL, about 2,000 ng·h/mL to about 8,000 ng·h/mL, about 2,000 ng·h/mL to about 9,000 ng·h/mL, about 2,000 ng·h/mL to about 10,000 ng·h/mL, about 3,000 ng·h/mL to about 4,000 ng·h/mL, about 3,000 ng-h/mL to about 5,000 ng·h/mL, about 3,000 ng·h/mL to about 6,000 ng·h/mL, about 3,000 ng·h/mL to about 7,000 ng·h/mL, about 3,000 ng·h/mL to about 8,000 ng·h/mL, about 3,000 ng·h/mL to about 9,000 ng·h/mL, about 3,000 ng·h/mL to about 10,000 ng·h/mL, about 4,000 ng·h/mL to about 5,000 ng·h/mL, about 4,000 ng·h/mL to about 6,000 ng·h/mL, about 4,000 ng·h/mL to about 7,000 ng·h/mL, about 4,000 ng·h/mL to about 8,000 ng·h/mL, about 4,000 ng·h/mL to about 9,000 ng·h/mL, about 4,000 ng·h/mL to about 10,000 ng·h/mL, about 5,000 ng·h/mL to about 6,000 ng·h/mL, about 5,000 ng·h/mL to about 7,000 ng·h/mL, about 5,000 ng-h/mL to about 8,000 ng·h/mL, about 5,000 ng·h/mL to about 9,000 ng·h/mL, about 5,000 ng·h/mL to about 10,000 ng·h/mL, about 6,000 ng·h/mL to about 7,000 ng·h/mL, about 6,000 ng·h/mL to about 8,000 ng·h/mL, about 6,000 ng·h/mL to about 9,000 ng·h/mL, about 6,000 ng·h/mL to about 10,000 ng·h/mL, about 7,000 ng·h/mL to about 8,000 ng·h/mL, about 7,000 ng·h/mL to about 9,000 ng·h/mL, about 7,000 ng·h/mL to about 10,000 ng·h/mL, about 8,000 ng·h/mL to about 9,000 ng·h/mL, about 8,000 ng·h/mL to about 10,000 ng·h/mL, or about 9,000 ng-h/mL to about 10,000 ng·h/mL. In some embodiments, the level of average plasma concentration and/or average plasma exposure (AUC) of sulcardine or a pharmaceutically acceptable salt thereof in a given patient is about 1,000 ng·h/mL, about 2,000 ng·h/mL, about 3,000 ng·h/mL, about 4,000 ng·h/mL, about 5,000 ng·h/mL, about 6,000 ng·h/mL, about 7,000 ng·h/mL, about 8,000 ng·h/mL, about 9,000 ng·h/mL, or about 10,000 ng·h/mL. In some embodiments, the level of average plasma concentration and/or average plasma exposure (AUC) of sulcardine or a pharmaceutically acceptable salt thereof in a given patient is at least about 1,000 ng·h/mL, about 2,000 ng·h/mL, about 3,000 ng·h/mL, about 4,000 ng·h/mL, about 5,000 ng·h/mL, about 6,000 ng·h/mL, about 7,000 ng·h/mL, about 8,000 ng·h/mL, or about 9,000 ng·h/mL. In some embodiments, the level of average plasma concentration and/or average plasma exposure (AUC) of sulcardine or a pharmaceutically acceptable salt thereof in a given patient is at most about 2,000 ng·h/mL, about 3,000 ng·h/mL, about 4,000 ng·h/mL, about 5,000 ng·h/mL, about 6,000 ng·h/mL, about 7,000 ng·h/mL, about 8,000 ng·h/mL, about 9,000 ng·h/mL, or about 10,000 ng·h/mL.

In some embodiments, the level of average plasma concentration and/or average plasma exposure (AUC) of sulcardine or a pharmaceutically acceptable salt thereof in a patient is about 1,000 ng·h/mL to about 30,000 ng-h/mL. In some embodiments, the level of average plasma concentration and/or average plasma exposure (AUC) of sulcardine or a pharmaceutically acceptable salt thereof in a given patient is about 1,000 ng·h/mL to about 25,000 ng·h/mL, about 1,000 ng·h/mL to about 20,000 ng·h/mL, about 1,000 ng·h/mL to about 15,000 ng·h/mL, about 1,000 ng·h/mL to about 10,000 ng·h/mL, about 1,000 ng·h/mL to about 7,500 ng·h/mL, about 1,000 ng·h/mL to about 5,000 ng·h/mL, about 1,500 ng·h/mL to about 30,000 ng·h/mL, about 25,000 ng·h/mL to about 20,000 ng·h/mL, about 1,500 ng·h/mL to about 15,000 ng·h/mL, about 7,500 ng·h/mL to about 5,000 ng·h/mL, about 2,000 ng·h/mL to about 30,000 ng·h/mL, about 2,000 ng·h/mL to about 25,000 ng·h/mL, about 2,000 ng·h/mL to about 20,000 ng·h/mL, about 2,000 ng·h/mL to about 15,000 ng·h/mL, about 2,000 ng·h/mL to about 10,000 ng·h/mL, about 2,000 ng·h/mL to about 7,500 ng·h/mL, about 2,000 ng·h/mL to about 5,000 ng·h/mL, about 5,000 ng·h/mL to about 30,000 ng·h/mL, about 5,000 ng·h/mL to about 25,000 ng·h/mL, about 5,000 ng·h/mL to about 20,000 ng·h/mL, about 5,000 ng·h/mL to about 15,000 ng·h/mL, about 5,000 ng·h/mL to about 12,500 ng·h/mL, or about 5,000 ng·h/mL to about 10,000 ng·h/mL. In some embodiments, the level of average plasma concentration and/or average plasma exposure (AUC) of sulcardine or a pharmaceutically acceptable salt thereof in a given patient is about 1,000 ng·h/mL, about 2,000 ng·h/mL, about 6,000 ng·h/mL, about 7,500 ng·h/mL, about 10,000 ng·h/mL, about 12,500 ng·h/mL, about 15,000 ng·h/mL, about 17,500 ng·h/mL, about 20,000 ng·h/mL, about 22,500 ng·h/mL, about 25,000 ng·h/mL, about 30,000 ng·h/mL, about 35,000 ng·h/mL, or more.

In some embodiments, abnormal heartbeat is fibrillation or heart flutter. In some embodiments, the abnormal heartbeat is fibrillation. In some embodiments, fibrillation is atrial fibrillation (AF) or ventricular fibrillation (VF). In some embodiments, the AF is acute AF. In some embodiments, the AF is paroxysmal AF. In some embodiments, the AF is recurrent AF. In some embodiments, the abnormal heartbeat is atrial flutter.

### EXAMPLES

Aspects of the present disclosure may be further understood in light of the following examples, which should not be construed as limiting the scope of the present disclosure in any way.

### Abbreviation

AF atrial fibrillation
CI confidence interval
Cmax maximum observed blood plasma concentration
ECG electrocardiogram
IV intravenous
JTp interval from the J point to the peak of the T wave
JTpc JTp interval corrected using Fridericia's formula (JTp=JTpF= (JTp/(RR)^{1/3})
msec millisecond
QRS interval from the end of the PR interval to the end of the S wave
QT interval from beginning of the QRS complex to the end of the T wave
QTc QT interval corrected for the effect of increasing heart rate (generically refers to one of many possible formulae used to correct for the QT variability due to changes in heart rate, including QTcB, QTcF, etc.)
QTcF QT interval corrected for heart rate using Fridericia's formula (QTcF= (QT/(RR)^{1/3})
RR Peak to peak R interval
SD standard deviation
SR Sinus rhythm
TdP Torsades de Pointes
Tmax time to achieve the maximum observed plasma concentration
VF ventricular fibrillation

### Example 1-JTp Inhibition with Sulcardine

HBI-3000 (sulcardine sulfate) is a multi-ion channel blocker with relatively balanced in vitro inhibitory potency on peak sodium (I_{Na-Peak}), late sodium (I_{Na-Late}), L-type calcium (I_{Ca.L}), and rapidly activating delayed-rectifier (I_{Kr}, hErg) currents. HUYABIO International LLC is developing HBI-3000, and other salts of sulcardine, as anti-fibrillatory drugs, e.g., for treatment of atrial fibrillation (AF).

The therapeutic effect of I_{Kr} (hERG) inhibition includes QT prolongation, which, when excessive, can cause malignant ventricular arrhythmias. QT prolongation is typically associated with a proportional lengthening of the J to T peak (JTp) interval, which corresponds to the early repolarization phase of the cardiomyocyte action potential. As shown herein, sulcardine protects against excessive QT prolongation with uniquely strong JTp shortening. This effect represents a reduction in early repolarization time, likely mediated by sulcardine's inhibition of I_{Na-late} and I_{Ca.L}. JTp interval reduction is the 12-lead EKG manifestation of a decrease in duration in the early repolarization period, which is the cellular mechanism underlying the protective mechanism of sulcardine.

Objective: The JTp mechanism, pharmacokinetics (PK), electrocardiogram (ECG), and echocardiogram changes, safety, and drug-drug interaction (DDI) of a single intravenous (IV) infusion of sulcardine (as sulcardine sulfate) was explored.

Methods: An open-label Phase 1 study evaluated ECG changes resulting from a 350 mg, 30-minute infusion of sulcardine (as the sulfate salt HBI-3000) in 39 healthy volunteers who were in SR. Triplicate electrocardiograms (ECGs) were extracted from continuous 12-lead Holter ECG recordings at baseline and 10 time points thereafter. Mean baseline-subtracted and heart rate-corrected QT (dQTcF), JTp (dJTpc), and other ECG changes were calculated at each time point. Left ventricular ejection fraction (LVEF) was assessed at baseline, 30, and 120 minutes after the start of the infusion by transthoracic echocardiography (TTE). Potential DDI between sulcardine and the cytochrome P450 CYP2D6 enzyme inhibitor paroxetine was investigated in period 2 of the study.

Results: Sulcardine's electrocardiogram (ECG) profile indicates a unique balance of inward and outward current block expected to protect against proarrhythmic risk. ECG parameter changes proportional to sulcardine plasma concentrations were observed, including modest observed QTcF prolongation (FIG. 4, middle slope, confidence band, “Observed dQTcF”) as expected for an I_{Kr} inhibitor. However, sulcardine did not increase the JTpc typically seen with I_{Kr} blockers, even those with mitigating inward channel inhibition that reduce but don't reverse the JTp increase at therapeutic doses.

Conclusions: Acute IV HBI-3000 (sulcardine) administration (30 min.) was well tolerated and resulted in no clinically significant safety findings, LVEF depression, or DDI with CYP2D6. The balance of sulcardine's inward and outward current inhibition provides the therapeutic benefit of modest QT prolongation (via I_{Kr} block) with a simultaneous reduction in the JTpc interval, reflecting reduced early repolarization time (via I_{Na-late} and I_{Ca.L}. block). This intrinsic mechanism may protect against the proarrhythmic risk of excessive QT prolongation caused by other I_{Kr} blockers used in treating AF. Preliminary results of a Phase 2 trial of this promising drug have been reported in an abstract (Roy et al, 2025).

### Example 2-Safety and Efficacy of HBI-3000 (Sulcardine Sulfate), a Multichannel Blocker for Patients with Non-Permanent Atrial Fibrillation

Introduction: Atrial fibrillation (AF) patients need a safe, fast, effective alternative to electrical cardioversion to restore sinus rhythm (SR). HBI-3000 (sulcardine sulfate) is a novel bioinspired new drug with promising antiarrhythmic properties, excellent tolerability and a unique mechanism limiting QT prolongation and risk of inducing lethal arrhythmias. We assessed the safety and efficacy of a single 30-minute (min) IV infusion of sulcardine sulfate to convert AF to SR in the hospital setting.

Methods: An open-label phase 2 dose-escalation study investigated a 30-min IV HBI-3000 infusion to convert recent-onset AF (>2 and <72 hours (h) duration) in presenting patients. Dose levels assessed were 200 mg, 350 mg, and 500 mg. After infusion initiation, serial vital signs, transthoracic echocardiograms, PK drug levels, 6 h continuous telemetry, and 24 h 12-lead Holter ECG monitoring were assessed, with a 30 day safety follow-up. Primary efficacy endpoint was proportion of patients converting to SR within 120 min of infusion start.

Results: Analysis included 28 Per Protocol patients (median, range) (age 62.5 years, 28-80; 60.7% males; weight 90.5 kg, 57-224; BMI 30.2 kg/m2, 21.6-77.5). Median AF time was 16.1 h (range 5.2-71.1) before infusion start; 64% new-onset AF, 36% paroxysmal AF. No Serious Adverse Events or drug-related arrhythmias were seen through 30-day follow-up. HBI-3000 infusion had only minor impact on mean HR and BP, and an insignificant impact on cardiac contractility measured by serial LVEF. Across all BMI, AF conversion rates for 200 mg, 350 mg, and 500 mg doses were 37.5%, 50%, and 30%, respectively. While obese patients (BMI≥30 kg/m2) had consistently low rates at all doses (w13%), non-obese patient conversion rates were 60%, 80%, and 66.7% at 200 mg, 35 0 mg, and 500 mg, respectively. Mean time to SR conversion from infusion start for all patients was 45.1 min, with 91.6% of conversions within 90 min or less.

Sulcardine strongly reduced JTpc (FIG. 5, “Observed dJTpc”), indicating faster early repolarization that may protect from excessive QT prolongation. The observed dQTcF was lower than expected, (FIG. 5, second from top, “Observed dQTcF”), i.e., in the absence of JTpc reduction, the QTcF change would have been significantly higher (FIG. 5, upper, “Calculated dQTcF if no dJTpc effect”)—by as much as 21 msec at the highest concentrations. No clinically significant safety findings, arrhythmias, or clinically significant LVEF changes were observed. Sulcardine had minimal CYP2D6 metabolic interactions in the DDI portion of the study.

Sulcardine IV infusion was well tolerated in acute-onset AF patients and demonstrated a clinically meaningful therapeutic effect.

### Conclusion and Incorporation by Reference

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed:

1. A method of treating atrial fibrillation (AF), atrial flutter or other arrhythmia, comprising administering to a human subject a dose of sulcardine, wherein administering the dose of sulcardine to the human subject causes a sulcardine Cmax in the human subject of about 100 ng/ml to about 35,000 ng/ml and a QTc prolongation of 0 ms to 60 ms.

2. The method of claim 1, wherein administering the dose of sulcardine causes a QTc of less than about 500 ms.

3. The method of claim 1, wherein the QTc prolongation is less than 50 ms.

4. The method of claim 1, wherein the QTc prolongation is less than 40 ms.

5. A method of protecting against QTc prolongation in a human subject, comprising administering to the human subject an effective amount of a compound, wherein the compound has a JTpc interval shortening activity and the human subject has an electrocardiogram (ECG) having a QTc.

6. The method of claim 5, wherein the JTpc interval shortening activity comprises inhibiting, in a cardiomyocyte, an inward sodium current, an inward calcium current, or both.

7. The method of claim 6, wherein the inward sodium current comprises both an inward early sodium current and a late inward sodium current.

8. The method of claim 5, wherein administering the compound to the human subject causes the QTc prolongation to be less than about 60 ms.

9. The method of claim 5, wherein the QTc is less than about 500 ms.

10. A method of protecting against QTc prolongation in a human subject, comprising administering to the human subject an effective amount of a compound, wherein the compound inhibits one or both of a late sodium inward ion current and an L-type calcium current in cardiomyocytes, thereby shortening a JTpc interval.

11. The method of claim 10, wherein the compound inhibits both the late sodium inward ion current and the L-type calcium current in cardiomyocytes.

12. The method of claim 11, wherein the QTc prolongation is less than about 60 ms.

13. The method of claim 12, wherein the QTc prolongation is less than 50 ms.

14. The method of claim 13, wherein the QTc prolongation is less than 40 ms.

15. The method of claim 10, wherein the human subject has a QTc of less than about 500 ms.

16. The method of claim 10, wherein the compound is sulcardine or a pharmaceutically acceptable salt of sulcardine.

17. The method of claim 16, wherein the effective amount of sulcardine, or a pharmaceutically acceptable salt of sulcardine, is about 200 mg to about 500 mg based on sulcardine sulfate.

18. The method of claim 17, wherein the effective amount of the compound causes a maximum blood plasma concentration (Cmax) of up to 5,000 ng/mL.
